(19) United States
(12) Patent Application Publication
(10) Pub. No.: US 2023/0348850 A1
(43) Pub. Date: Nov. 2, 2023

(54) CELL CULTURE PROCESSES

(71) Applicant: UCB BIOPHARMA SRL, BRUSSELS (BE)

(72) Inventors: BASSEM BEN YAHIA, BRUSSELS (BE); ANTOINE PHILIPPE THOMAS PIEDNOIR, BRUSSELS (BE)

(21) Appl. No.: 18/021,820

(22) PCT Filed: Aug. 19, 2021
(86) PCT No.: PCT/EP2021/073094

§ 371 Date: Feb. 17, 2023

(30) Foreign Application Priority Data
Aug. 20, 2020 (GB) 2012991.2

(51) Int. Cl. C12N 5/073; C07K 16/06; C12N 5/00

(52) U.S. Cl. CPC C12N 5/0603; C07K 16/06; C12N 5/0018; C07K 2317/14

(57) ABSTRACT

The present invention belongs to the field of the manufacture of recombinant proteins, particularly proteins, such as antibodies.

### FIELD OF INVENTION

The present invention belongs to the field of the manufacture of recombinant proteins, particularly proteins, such as antibodies.

### BACKGROUND OF THE INVENTION

Development of recombinant proteins as therapeutic proteins, such as therapeutic antibodies, requires production of the recombinant proteins at an industrial scale. In order to achieve this, different expression systems, both prokaryotic and eukaryotic systems, may be employed. Over the past two decades, however, the majority of the approved therapeutic proteins have been manufactured through mammalian cell cultures and such systems remain the preferred expression system for producing a large quantity of recombinant proteins for use in humans.

Cell culture conditions, such as the composition of the medium (Kshirsagar R., et al., 2012; US20130281355; WO2013158275) and the growing conditions, including pH and temperature (WO2011134919) have been shown to impact the yield and the quality attributes of therapeutic proteins. Over the last 30 years, much effort has been dedicated to establishing the basic parameters of cell culture, media and recombinant protein expression with much focus of the research dedicated to reaching optimal cell growth through changes of the composition of the cell culture media (see e.g. Hecklau C, et al., 2016; Zang L. et al., (2011)), operating conditions and development of large bioreactors.

Some components present at high concentration in feed media tend to precipitate during storage (until said media are added in the bioreactor), especially when the pH of said medium is around neutrality. Such a precipitation prior to use is not desirable: indeed, it may have an impact on the exact composition of the medium (as the amount of components in solution/in the precipitate will be unknown). WO2008013809, which relates to chemically concentrated feed media (between 10× to 100×), discloses that salts typically precipitate when dissolved together at certain pH values, such as at pH above 5.8, or yet that other components such as folic acid require a pH of 8.6 for solubilization. WO2008141207 provides stable feed media containing cysteine, tyrosine and optionally cystine, and further containing pyruvate as a stability agent towards components that are difficult to solubilise at high concentration (such as tyrosine or cysteine). WO2011133902 proposes to supplement the concentrated feed media with small peptides having two to six amino acids (such as alanyl tyrosine and/or alanyl cysteine and/or alanyl cystine dimer) in order to limit the risk of precipitation of said feeds.

Yet, there remains the need to provide further improved feed media to be used in the context of cell culture processes for the production of therapeutic proteins, while having a minimal impact on yield and protein heterogeneity.

### SUMMARY OF THE INVENTION

In a first aspect, the invention provides a process for culturing mammalian cells expressing a recombinant protein, wherein the process comprises the steps of culturing the mammalian cells in a culture medium, and supplementing the cell culture during a production phase with at least one feed medium, wherein the pH of this at least one feed medium is from about 5.0 to about 6.3.

In a second aspect, the invention provides a process for producing a recombinant protein, wherein the process comprises the steps of culturing mammalian cells expressing said recombinant protein in a culture medium, and supplementing the cell culture during a production phase with at least one feed medium, wherein the pH of this at least one feed medium is from about 5.0 to about 6.3. In a third aspect, the invention provides a process for reducing or preventing precipitation in a feed medium, wherein the process comprises the step of culturing mammalian cells expressing a recombinant protein in a culture medium, and supplementing the cell culture during a production phase with at least one feed medium, wherein the pH of this at least one feed medium is from about 5.0 to about 6.3.

In a fourth aspect, the invention relates to a feed medium for use in any of the processes herein described, wherein the pH of this at least one feed medium is from about 5.0 to about 6.3 In a fifth aspect, the invention describes a recombinant protein produced by any one of the processes according to the present invention. In the context of any one of these aspects, the feed medium is a main feed medium, such as a concentrated main feed medium.

### Definitions

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the term “and/or” used in a phrase such as “A and/or B” herein is intended to include “A and B”, “A or B”, “A”, and “B”.

As used in the specification and claims, the term “cell culture” or “culture” is meant the growth, propagation and/or maintenance of cells in vitro, i.e. outside of an organism or tissue. Suitable culture conditions for mammalian cells are known in the art, such as taught in Cell Culture Technology for Pharmaceutical and Cell-Based Therapies (2005). Mammalian cells may be cultivated in suspension or while attached to a solid substrate.

The terms “cell culture medium,” “culture medium”, “medium,” and any plural thereof, refer to any medium in which cells of any type can be cultivated. A “basal medium” refers to a cell culture medium that contains all of the essential ingredients useful for cell metabolism. This includes for instance amino acids, lipids, carbon source, vitamins and mineral salts. DMEM (Dulbeccos' Modified Eagles Medium), RPMI (Roswell Park Memorial Institute Medium) or medium F12 (Ham's F12 medium) are examples of commercially available basal media. Other suitable media have e.g. been described in WO9808934 and US20060148074 (both incorporated herein in their entirety). Further suitable commercially available media include, but are not limited to, AmpliCHO CD medium, Dynamis™ Medium, EX-CELLO Advanced™ CHO Fed-batch System, CD FortiCHO™ medium, CP OptiCHO™ medium, Minimum Essential Media (MEM), BalanCD® CHO Growth A Medium, ActiPro™ medium, DMEM—Dulbecco's Modified Eagle Medium and RPMI-1640 medium. Alternatively, said basal medium can be a proprietary medium, also herein called “chemically defined medium” or “chemically defined culture medium”, in which all of the components can be described in terms of the chemical formulas and are present in specific concentrations. The culture medium is preferably free of proteins and free of serum, and can be supplemented by any additional compound(s) such as amino acids, salts, sugars, vitamins, hormones, growth factors, depending on the needs of the cells in culture.

The term “feed medium” or “feed” (and plural thereof) refers to a medium which is added during culture to replenish the nutrients which are consumed. The feed medium can be a commercially available feed medium or a proprietary feed medium (herein alternatively named “defined feed medium” or “chemically defined feed medium”). Suitable commercially available feed media include, but are not limited to, Cell Boost™ supplements, EfficientFeed™ supplements, ExpiCHO™ Feeds. Alternatively, said feed medium can be a proprietary feed medium in which all of the components can be described in terms of the chemical formulas and are present in specific concentrations. A feed medium is typically concentrated, compared to a basal medium, in order not to increase to a high level the final volume of the culture. Such a feed medium can contain most of the components of the cell culture medium at, for example, about 1.5×, 2×, 5×, 6×, 7×, 8×, 9×, 10×, 12×, 14×, 16×, 20×, 30×, 50×, 100×, 200× or even 500× of their normal amount in a basal medium. Proprietary feed media are typically in powder. Commercial feeds are either liquid or in powder. When feeds are already in liquid form, they are used as such, according to the leaflet. Feeds which are in powder need to be solubilised, in water for instance, before use. They are supposed to be solubilised in a given amount of water (e.g. 100 g in 1 L of water, see FIG. 1A). However, feeds in powder can be further concentrated. In such a case, they will be solubilised using less liquid than normally needed for said quantity (e.g. 200 g in 1 L of water as shown in FIG. 1B). Liquid commercial feeds or feeds in powder that are prepared according to the standard protocol are herein also called “normal” feed. Liquid commercial feeds or feeds in powder that are prepared according to a concentrated process are herein called “concentrated feed”.

Different feed media of different compositions can be added throughout the culture process. For instance, three different feed media can be used during the same process: one feed medium consisting of the carbon source (e.g. glucose), one feed medium comprising most of the nutrients which are consumed (this feed is, herein, also referred to as “main feed”, “main feed medium” or “at least one feed medium”), and a further feed medium comprising some further nutrients for instance when this nutrients present aggregation/stability issues (such as for instance cysteine and/or cystine and/or tyrosine).

The term “bioreactor” refers to any system in which cells can be cultivated. It includes but is not limited to flasks, static flasks, spinner flasks, tubes, shake tubes, shake bottles, wave bags, bioreactors, fibre bioreactors, and stirred-tank bioreactors with or without microcarriers. Alternatively, this term also includes microtiter plates, capillaries or multi-well plates. Any size of bioreactor can be used, for instance from 1 millilitre (1 mL, very small scale) to 20000 litres (20000 L or 20 KL, very large scale), such as 0.1 mL, 0.5 mL, 1 mL, 5 mL, 0.01 L, 0.1 L, 1 L, 2 L, 5 L, 10 L, 50 L, 100 L, 500 L, 1000 L (1 KL), 2000 L (2 KL), 5000 L (5 KL), 10000 L (10 KL), 15000 L (15 KL) or 20000 L (20 KL).

The term “fed-batch culture” refers to a process of growing cells, where there is a bolus (or several boli) or continuous feed medium (or feed media) added (note that “addition” is also named “supplementation” in the context of this invention) to replenish the nutrients which are consumed, without removal of any medium. Feed(s) can be added according to a predetermined schedule of, for example, every day, once every two days, once every three days, etc. Alternatively, should the feeding be continuous, the feeding rate can be varied throughout the culture. This cell culture technique has the potential to obtain high cell densities in the order of greater than 10×10⁶ to 30×10⁶ cells/ml, depending on the media formulations, cell line, and other cell growth conditions. A biphasic culture condition can be created and sustained by a variety of feed strategies and media formulations.

When using the processes and/or cell culture techniques of the present invention, in mammalian cells, the recombinant proteins are generally directly secreted into the culture medium. Once said protein is secreted into the medium, supernatants from such expression systems can be first harvested and clarified, in order to start isolating the protein of interest and concentrate if before it is purified and formulated.

The term “production phase” according to the present invention comprises that stage of cell culturing during the process for manufacturing a recombinant protein when the cells express (i.e. produce) the recombinant polypeptide(s). The production phase typically begins when the titre of the desired recombinant protein increases and/or when cell growth has essentially ended and ends with harvest of the cells (or the cell culture fluid or supernatant) when the recombinant protein production has essentially ended. The cells may be maintained in production phase until a desired cell density or desired recombinant protein titre is reached. For instance, the cells are maintained in the subsequent production phase until the titre of the recombinant protein reaches a maximum. Alternatively, the culture may be harvested prior to this point, depending on the production requirement of the skilled person or the needs of the cells themselves. Typically, at the beginning of the production phase, the cell culture is transferred from a pre-production vessel (N−1 vessel) to a production vessel (N vessel), such as a bioreactor. In the N−1 vessel, cells can be grown according to any technics from the art, such as in perfusion mode, batch mode or fed-batch mode. Harvest is the step during which the cell culture fluid is removed from the production vessel, in order for the recombinant protein, e.g. the recombinant antibody, to be recovered and purified in subsequent steps.

As used herein, “cell concentration” (also known as “cell density”) refers to the number of cells in a given volume of culture medium. “Viable cell concentration” (or “VCC”) refers to the number of living cells in a given volume of culture medium. This is determined by standard viability assays.

The term “viability”, or “cell viability” refers to the ratio between the total number of viable cells and the total number of cells in culture. Although the viability is typically acceptable as long as it does not go below a 60% threshold compared to the start of the culture, the acceptable threshold can be determined on a case by case basis. Viability is often used to determine time for harvest. For instance, in fed-batch culture, harvest can be performed once viability reaches at 60% or after about 14 days (typically 14 days+/−1 day) in culture.

The wording “titre” refers to the concentration of the recombinant protein of interest in solution. This is determined by standard tire assays, such as serial dilutions combined with a detection method (colorimetric, chromatographic etc.), with a CEDEX or protein A high-pressure liquid chromatography (HPLC), Biacore C® or ForteBIO Octet® methods, as used in the example section.

The term “higher titre” or “higher productivity”, and equivalents thereof, means that the titre or the productivity is increased by at least 10% when compared to the control culture condition. The titre or specific productivity will be considered as maintained if it is in the range of −10% to 10% compared to the control culture condition. The terms “lower titre” or “lower productivity”, and equivalents thereof, means that the titre or the productivity is decreased by at least 10% when compared to the control culture condition.

Precipitation of elements composing the feed medium (in the context of this invention it is also referred to precipitation of the feed) may occurred after preparation or/and storage step. Precipitation can be visually assessed as small solid particles in solution (in solution and/or sedimenting as particles at the bottom of the container). Said assessment is well within the knowledge of the skilled person. The term “reduction of precipitation” is to be understood as the decrease of sedimented precipitates or/and precipitates in feed medium, as assessed visually for instance, when compared with the precipitation observed under control conditions. The term “prevention of precipitation” is to be understood as the absence of sedimented precipitates or/and precipitates in feed medium, as assessed visually for instance.

The term “heterogeneity” as used herein refers to differences between individual molecules, e.g. recombinant proteins, in a population of molecules produced by the same manufacturing process, or within the same manufacturing batch. Heterogeneity can result from incomplete or inhomogeneous modifications of the recombinant polypeptides, e.g. due to post-translational modifications of the polypeptide or to misincorporation during transcription or translation. Post-translational modifications can e.g. be the result of deamination reactions and/or oxidation reactions and/or covalent addition of small molecules such as glycation reactions and/or isomerization reactions and/or fragmentation reactions and/or other reactions and also include variation on the glycation patterns. The chemo-physical manifestation of such heterogeneity leads to various characteristics in the resulting recombinant polypeptide preparations which include, but are not limited to, charge variant profile, colour or colour intensity and molecular weight profile. When measuring the isoforms of the recombination proteins, besides the main charge species are also measured the acidic isoforms (APG) and the basic isoforms (BPG). The main charge species represents the isoform of the recombinant protein that one wishes to obtain.

The term “recombinant protein” means a protein produced by recombinant technics. Recombinant technics are well within the knowledge of the skilled person (see for instance Sambrook et al., 1989, and updates). The term “protein” can be for instance a cytokine, a growth factor, a hormone, an antibody or a fusion protein comprising a domain or other fragments of an antibody. The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments and in particular to antigen-binding fragments. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)2 (also referred to as TrYbe®, see WO2015197772 for instance). Antibody fragments as defined above are known in the art.

### DETAILED DESCRIPTION OF THE INVENTION

Typically, aqueous feed solutions are prepared (dissolution of a powder until the expected concentration is reached in the liquid; the pH is adjusted to the target value; see FIG. 1) before the start of the production process and are stored in a container (e.g. bag or feed tank) until they are added to the production bioreactor. The storage can be done at low temperature (e.g. 2-8° C.) until the bag or the tank is connected to the production bioreactor. From this moment, the storage is typically performed at room temperature. It is understood that as a production process typically lasts about 14 days, the main feed is stored up to 14 days and possibly longer should it have been prepared well before the start of the culture. Precipitation of feed media in their container (such as in a bag or in a tank) has been observed, typically by visual observation.

The present invention generally relates to processes for the production of recombinant proteins in mammalian cells. In particular, the invention is based on the finding from the inventors that by lowering the pH of the main feed to be added in the frame of a fed-batch process it was possible to avoid, or at least reduce, the precipitation of said main feed throughout the cultivation process without impacting the overall process performances (e.g. as assessed in term of VCC or titre).

In another embodiment, the invention provides a process for culturing mammalian cells expressing a recombinant protein, wherein the process comprises the steps of culturing the mammalian cells in a culture medium, and supplementing the cell culture during a production phase with at least one feed medium, wherein the pH of this at least one feed medium is from about 5.0 to about 6.3. In another embodiment, the invention provides a process for producing a recombinant protein, wherein the process comprises the steps of culturing mammalian cells expressing said recombinant protein in a culture medium, and supplementing the cell culture during a production phase with at least one feed medium, wherein the pH of this at least one feed medium is from about 5.0 to about 6.3.

In a further embodiment, herein described is a process for reducing or preventing precipitation in a feed medium, wherein the process comprises the steps of culturing mammalian cells expressing a recombinant protein in a culture, and supplementing the cell culture during a production phase with at least one feed medium, wherein the pH of this at least one feed medium is from about 5.0 to about 6.3.

In the context of the invention as a whole, the processes for producing a recombinant protein, for culturing mammalian cells expressing a recombinant protein or for reducing or preventing precipitation in a feed medium, comprise the following main steps:
(i) inoculating the mammalian cells in a culture medium (such as a basal medium) in a bioreactor (such as a production bioreactor),
(ii) progressing the culture through the production phase wherein the recombinant protein is produced by the mammalian cells, wherein, during said production phase, the cell culture is supplemented with at least one feed medium,

wherein the pH of this at least one feed medium is defined within specific ranges. Said feed medium is preferably the main feed medium and can be a concentrated feed medium (e.g. a concentrated main feed medium).

In another embodiment, the invention relates to a feed medium for use in any of the processes herein described, wherein the pH of this at least one feed medium is from about 5.0 to about 6.3. Depending on the overall strategy used for producing a recombinant protein, for culturing mammalian cells expressing a recombinant protein or for reducing or preventing precipitation in a feed medium, the feed medium according to the invention (i.e. having a pH from about 5.0 to about 6.3) can also be used during stages preceding the production phase, such as during the N−1 stage. In a further aspect, the invention describes a recombinant protein produced by any one of the processes according to the present invention.

In the context of the invention as a whole, the culture medium at the start of the culture (step (i)) is preferably a protein- and serum-free culture medium. Said protein- and serum-free culture medium can be commercially available or a chemically defined medium.

In the context of the invention as a whole, the at least one feed medium (also herein referred to as main feed medium) is preferably a protein- and serum-free feed medium and comprises all or most of the essential elements. If not included in the at least one feed medium, the carbon source can be brought via a different feed. The (at least one) feed medium can be a “normal feed”, prepared and used according to standard protocols (as disclosed in FIG. 1A) or can be a concentrated feed medium (e.g. a defined concentrated main feed medium, as disclosed in FIG. 1B or a commercial concentrated feed medium). Alternatively, also in the context of the invention as a whole, the (at least one) feed medium (also herein referred to as main feed medium) is preferably a protein- and serum-free feed medium and comprises all or most of the essential elements, but does not comprise any of the free amino acids: cysteine and/or cystine (both referred as Cys) and tyrosine (Tyr), as these amino acids are known to be difficult to solubilise and to stabilise at pH lower than about 8.0. The carbon source as well as Cys and Tyr can be brought via different feeds such as one feed consisting of the carbon source and 1) one feed consisting of Cys, Tyr or 2) two different feeds consisting of Cys and Tyr respectively. In the context of the invention as a whole, the at least one feed medium can be a “normal feed”, prepared and used according to standard protocols (as disclosed in FIG. 1A) or can be a concentrated feed medium (e.g. a defined concentrated main feed medium, as disclosed in FIG. 1B or a commercial concentrated feed medium). In yet another alternative, also in the context of the invention as a whole, the (at least one) feed medium (also herein referred to as main feed medium) is preferably a protein- and serum-free feed medium and comprises all or most of the essential elements, but does not comprise any of the free amino acids: cysteine and/or cystine (both referred as Cys), tryptophan (Trp) and tyrosine (Tyr). The carbon source as well as Cys, Tyr, Trp can be brought via different feeds such as one feed consisting of the carbon source and 1) one feed consisting of Cys, Tyr, Trp, 2) two feeds consisting of a combination of any two amino acids selected from of Cys, Tyr, Trp, the third amino acid being added in a separate feed or 3) three different feeds consisting of Cys, Tyr and Trp respectively. In the context of the invention as a whole, the at least one feed medium can be a “normal feed”, prepared and used according to standard protocols (as disclosed in FIG. 1A) or can be a concentrated feed medium (e.g. a defined concentrated main feed medium, as disclosed in FIG. 1B or a commercial concentrated feed medium).

In the context of the invention as a whole, the pH of the (at least one) feed medium is from about 5.0 to about 6.3, preferably from about 5.2 to about 6.2. The lower limits of the pH range can be selected for instance from any one of 5.20, 5.25, 5.30, 5.35, 5.40, 5.45, 5.50, 5.55, 5.60, 5.65, 5.70, 5.75, 5.80 or 5.85. The upper limits of the pH range can be selected for instance from any one of 6.00, 6.05, 6.10, 6.15 or 6.20. The pH of the feed medium according to the invention can for instance be 5.20, 5.25, 5.30, 5.35, 5.40, 5.45, 5.50, 5.55, 5.60, 5.65, 5.70, 5.75, 5.80, 5.85, 5.90, 5.95, 6.00, 6.05, 6.10, 6.15 or 6.20.

In the context of the invention as a whole, thanks to the lowering of the pH of the (at least one) feed medium, it was possible to avoid, or at least reduce, the precipitation of the main feed throughout the cultivation process without impacting the overall process performances.

In the context of the present invention, the production phase is carried out in a bioreactor (such as a production bioreactor), preferably with a volume of equal or more than 50 L, equal or more than 100 L, equal or more than 500 L, equal or more than 1,000 L, equal or more than 2,000 L, equal or more than 5,000 L, equal or more than 10,000 L or equal or more than 20,000 L. In other words, the mammalian cells producing the recombinant proteins are cultivated in a bioreactor (such as a production bioreactor), preferably with a volume of equal or more than 50 L, equal or more than 100 L, equal or more than 500 L, equal or more than 1,000 L, equal or more than 2,000 L, equal or more than 5,000 L, equal or more than 10,000 L or equal or more than 20,000 L.

In the context of the invention as a whole, suitable mammalian host cells (also named mammalian cells) include Chinese Hamster Ovary (CHO cells), lymphocytic cell lines, e.g., NSO myeloma cells and SP2 cells, COS cells, myeloma or hybridoma cells. In a preferred embodiment, the mammalian cell is a CHO. Suitable types of CHO cells may include CHO and CHO-K1 cells including dhfr-CHO cells, such as CHO-DG44 cells and CHO-DXB11 cells and which may be used with a DHFR selectable marker or CHOK1-SV cells which may be used with a glutamine synthetase selectable marker. The host cells are preferably stably transformed or transfected with expression vectors encoding the recombinant protein of interest.

In the context of the invention as a whole, the recombinant protein can be a cytokine, a growth factor, a hormone, a fusion protein (such as a protein comprising a domain or other fragments of an antibody), or an antibody. When the protein is an antibody, it is preferably an IgG such as IgG1, IgG2, IgG3 or IgG4.

The process of the invention optionally further comprises a step of recovering the recombinant protein from the cell culture medium, preferably at the end of the production (harvest step). Subsequently to the harvest, the recombinant protein may be purified, e.g. if the protein is an antibody, using Protein A chromatography. The process further optionally comprises a step of formulating the purified recombinant protein, e.g. into a formulation with a high protein concentration, such as a concentration of 10 mg/ml or more, e.g. 50 mg/ml or more, such as 100 mg/ml or more, e.g. 150 mg/ml or more. Without any limitation, the formulation can be a liquid formulation, lyophilised formulation or a spray-dried formulation.

### DESCRIPTION OF THE FIGURE

FIG. 1: A) Preparation of a “normal” feed from powder. B) Preparation of a “concentrated” feed from powder

FIG. 2: Viable Cell Concentration profiles of cells expressing mAb1

FIG. 3: Titre at day 13 and 14 for mAb1

FIG. 4: Viable Cell Concentration profiles of cells expressing mAb1

FIG. 5: Performances for producing mAb1

FIG. 6: Viable Cell Concentration profiles of cells expressing mAb2

FIG. 7: Titre at day 13 and 14 for mAb2

FIG. 8: Viable Cell Concentration profiles of cells expressing mAb3

FIG. 9: Titre at day 14 for mAb3

FIG. 10: Viable Cell Concentration profiles of cells expressing mAb4

FIG. 11: Titre at day 13 and 14 for mAb4

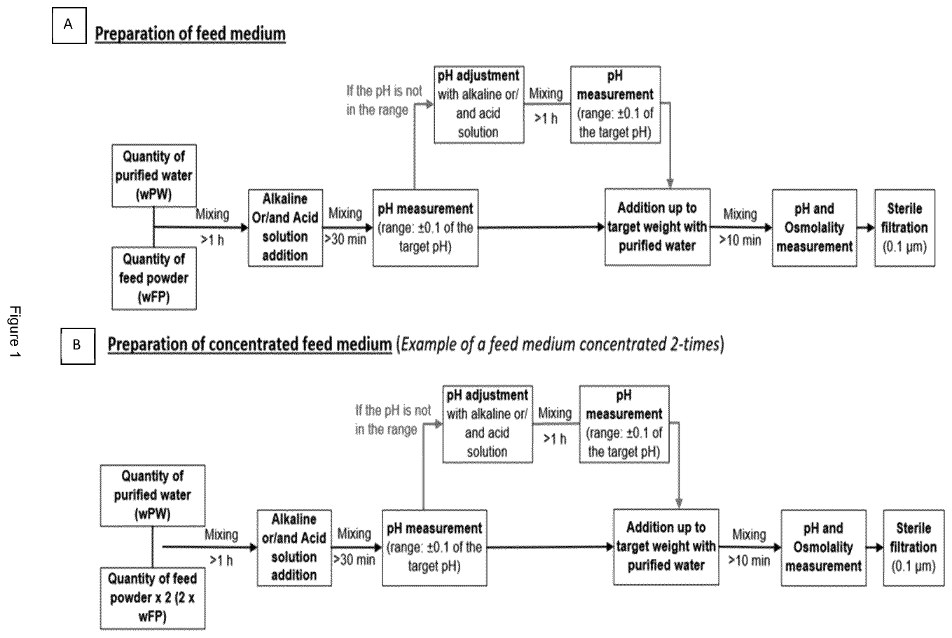

### EXAMPLES

Cell Line, Cell Culture and Experimental Procedure

CHO-DG44 cell lines were used. The cells were cultivated in 2 L stirred tank glass bioreactor (STR) with supply towers (C-DCUII, Sartorius Stedim Biotech) controlled by a multi-fermentation control system (MFCS, Sartorius Stedim Biotech) or in shake flasks. Four different production cell lines producing either mAb1, mAb2, mAb3 or mAb4 respectively, were used. mAb1 and mAb3 are IgG4 antibodies having respectively a pl of 8.3-8.7 and 7.70-7.90. mAb2 and mAb4 are Trybe® antibodies having a pl of 8.7-9.2.

The reactors were equipped with a 3-segment blade impeller. The cultivation start volume was adapted to ensure the cultivation end volume was optimal. The production bioreactors were seeded at target seeding density (TSD) in a basal chemically defined medium. The pH control of the production bioreactor was set to 7.0 with a dead band of 0.2 (pH 7.0±0.2). The pO2 target was set to 40-60% air saturation and controlled according to standard practice. The temperature was controlled at 36.8° C. with a dead band of 0.2 (36.8° C.±0.2).

A concentrated main feed was used for examples 1 to 3 and 5. This main feed (aqueous) was prepared by dissolution of the main feed powder until the desired concentration (concentrated about x2 compared to the standard protocol for this powder) was reached in the liquid and the pH was adjusted to the target pH. The non-concentrated feed used in example 4 was prepared by dissolution of the main feed powder according to the standard protocol for this powder (in order to reach a concentration of x1) and the pH was adjusted to the target pH. The bags with the main feeds were kept at room temperature during all the cultivation process. 48 hours after inoculation, continuous nutrient feeding (with a concentrated feed, named “main feed”) was started with a predetermined rate. A glucose bolus feed was added to the culture on demand, i.e. when the glucose concentration dropped below a given threshold (glucose concentrations were measured daily). As the main feed did not comprise any one of Cys, Tyr and Trp, these amino acids were added separately.

The production was operated in fed-experiment mode for 14 days, at room temperature. During this phase, the monoclonal antibody (mAb) is secreted into the medium. Samples were drawn daily to determine VCD, viability, off-line pH, pCO2, osmolality, glucose-lactate concentration, amino acid concentration and mAb concentration. Samples for the amino acid analysis were taken before the feed addition.

Analytical Methods

Cell were counted by using a VI-CELL® XR (Beckman-Coulter, Inc., Brea, CA) automated cell counting device operate that operated based on trypan blue exclusion. Glucose and lactate levels in the culture medium were determined using a Cedex Bio HT (Roche). A model 2020 freezing point osmometer (Advanced Instruments, Inc., Norwood, MA) was used for osmolality determination. Offline gas and pH measurements were performed with a model BioProfile pHOx® blood gas analyzer (Nova Biomedical Corporation, Waltham, MA). Metabolites concentrations are also daily determined using a CedexBioHT system (Roche). Product titre analysis was performed with the CEDEX or protein A high-pressure liquid chromatography (HPLC) with cell culture supernatant samples which were stored at −80° C. prior to analysis. The cell culture supernatant samples are purified with a Protein A purification on the AKTA Xpress system. The relative percentage of main isoform of the purified mAb is determined by Imaged Capillary Electrophoresis (ProteinSimple iCE3). Statistical analyses were performed using SAS software JMP 11 ©.

### Example 1—Low Feed pH Reduces Feed Precipitation while Maintaining Cell Culture Performances for Production Stage (N Stage)

For this experiment 2 L bioreactors were inoculated with CHO cells producing mAb1 at a seeding density of 3.75×10⁶ cells/mL. Inoculums for both bioreactors originated from the same N−1 bioreactor. In this experiment, three conditions were tested in fed-batch mode as described in the above experimental procedures. Bioreactors ID 1, 2 and 3 had the same feeding strategy but were fed with a main feed having two different pH: respectively 6.5, 6.0 and 5.5.

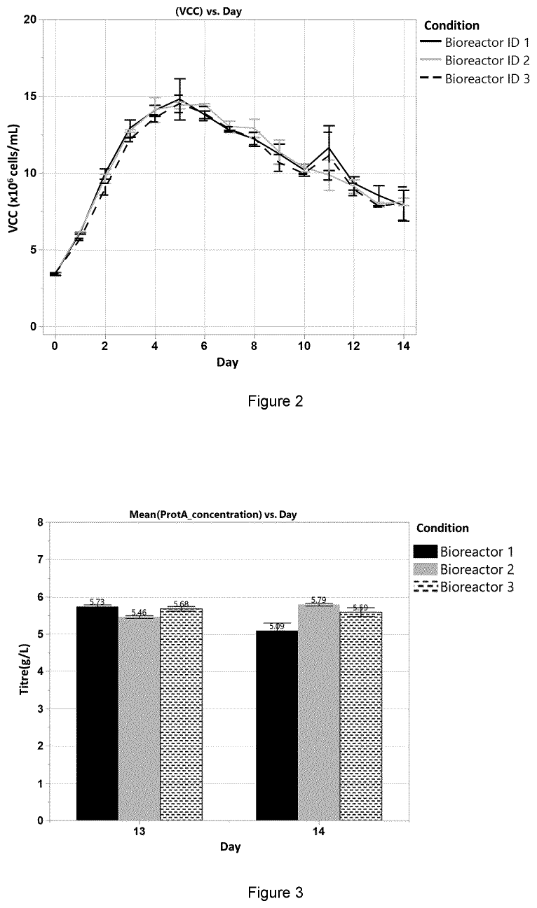

FIG. 2 shows similar trend at the three pH of the main feed. In addition, as reported in FIG. 3, lower pH of the main feed did not negatively impact the mAb titre. The cells grown under any of the tested conditions (i.e. pH 5.5, 6.0 and 6.5) display a similar titre of mAb1 whatever the day of the harvest (day 13 or day 14).

A significant impact was observed on main feed precipitation. As depicted in table 1, precipitation occurred with the main feed at pH 6.5 but not with the main feed having a lower pH (pH 6.0 and 5.5). Visual examinations were made at the end of the N stage (after disconnection of the main feed bottle from the bioreactors 1/2/3; pictures not shown). Cloudiness of the precipitated main feed solution was observed at pH 6.5. On the contrary, a clear/limpid solution was observed for the main feed solution at pH 6.0 or lower.

TABLE 1
Precipitation occurrence inside main feed bottle used
for supplementation during N stage

| | | Precipitation in the |
|---|---|---|
| Conditions | pH main feed | main feed bottle |
| Bioreactor ID 1 | 6.5 | Yes |
| Bioreactor ID 2 | 6.0 | No |
| Bioreactor ID 3 | 5.5 | No |

(Yes = precipitate occurrence; No = no precipitate occurrence).

It is noted that the pH of the culture was almost not impacted when the main feed at lower pH was used and was kept in the target range ±0.2 of the target pH 7.0.

Conclusion: Example 1 shows no difference between all conditions on process performances (as assessed via VCC and titre measurements). It was concluded that supplementing the culture with a main feed at a lower pH (compared to its standard pH, i.e. pH 6.5), during production process, did not impact process performances and could reduce and/or avoid precipitation during and/or after main feed supplementation.

### Example 2—Supplementation with a Main Feed at Low pH at Lame Scale to Produce mAb1

For this experiment, 2 L and 2000 L bioreactors were inoculated with CHO cells producing mAb1. The 2 L bioreactors were inoculated at a seeding density of 3.75×10⁶ cells/mL, whereas the 2000 L was inoculated at a seeding density of 3.40×10⁶ cells/mL. Inoculums for each bioreactor originated from the three different N−1 bioreactors. Two experimental conditions were tested in fed-batch process at different scales as described in the above experimental procedures. Bioreactor ID/4/5/6 had the same feeding strategy but were fed with main feeds having two different pH: respectively 6.5 and 6.0 (see Table 2).

TABLE 2
Experimental conditions for example 2

| Conditions | pH main feed | Scale |
|---|---|---|
| Bioreactor ID 4 | 6.0 | 2 L |
| Bioreactor ID 5 | 6.5 | 2 L |
| Bioreactor ID 6 | 6.0 | 2000 L |

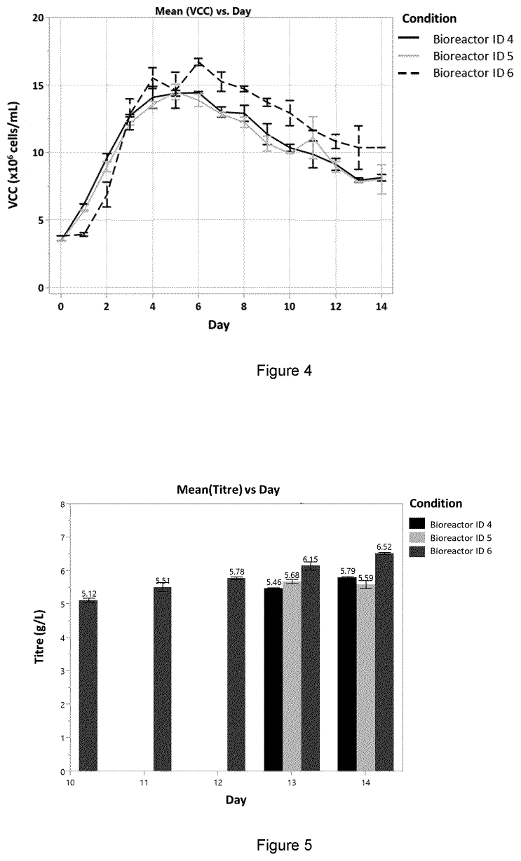

Cell growth profiles are depicted in FIG. 4. Cell growth for Bioreactors ID 4 and 5 presented similar trend. The Bioreactor ID 6 (large scale) showed a slight better cell growth compared to small scale conditions. Further, bioreactor ID 6 showed better titre (on days 13 and 14), compared to the other conditions (See FIG. 5). The results confirm that process at large scale lead to a better mAb1 production comparing data generated at 2 L scale. Furthermore, they confirm that there is no negative impact of supplementing a main feed at lower pH on overall mAb1 production.

As per Example 1, cloudiness of the precipitated main feed solution was observed at pH 6.5. On the contrary, a clear/limpid solution was observed for the main feed solution at pH 6.0, whatever the scale of production.

TABLE 3
Precipitation occurrence inside the main feed bottles (at day 14)

| | Precipitation in the main |
|---|---|
| Conditions | feed bottle |
| Bioreactor ID 4 | No |
| Bioreactor ID 5 | Yes |
| Bioreactor ID 6 | No |

Conclusion: Example 2 confirm the findings of example 1 and highlight that a main feed at lower pH, compared to its standard pH (i.e. pH 6.5), can be added to cell cultures during both small scale and large-scale processes to produce an antibody without having adverse effects on the overall process performances. Surprisingly, large scale results show an improvement of cell growth and final titre when compared to data generated at small scale.

### Example 3—Supplementation of Main Feed at Low pH to Produce mAb2

For this experiment, 2 L and 200 L bioreactors have been inoculated with CHO cells producing mAb2 at a seeding density of 2.25×10⁶ cells/mL. In this experiment, two experimental conditions were tested in fed-batch process at different scales as described in the above experimental procedures. Bioreactor ID 7/8/9 had the same feeding strategy but were fed with main feeds having two different pH: respectively 6.5 and 6.0 (see Table 4).

TABLE 4
Experimental conditions for example 4

| Conditions | pH Main feed | Scale |
|---|---|---|
| Bioreactor ID 7 | 6.5 | 2 L |
| Bioreactor ID 8 | 6.0 | 2 L |
| Bioreactor ID 9 | 6.0 | 200 L |

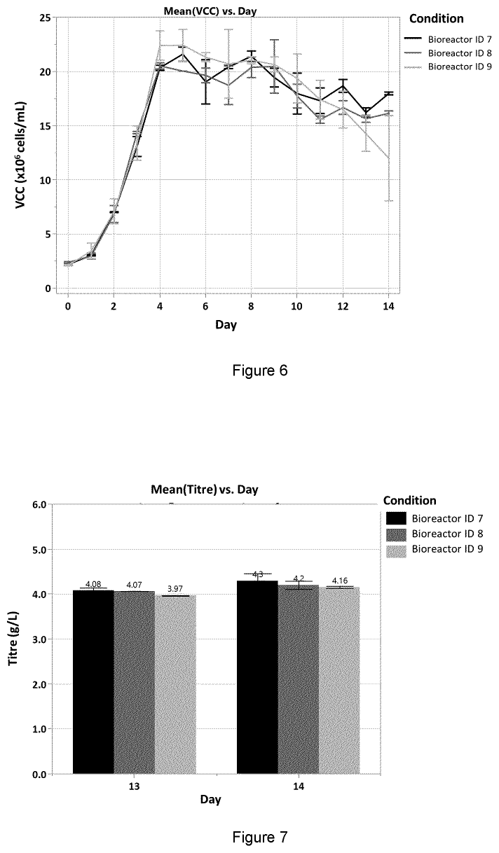

Cell growth profiles are shown in FIG. 6. Cell growth for both conditions and at both scales presented similar trend until day 13. Bioreactor ID 9 also shows a titre (on days 13 and 14), comparable to the ones of bioreactors 7 and 8 (See FIG. 7). The results confirm that there is no negative impact of adding a main feed at lower pH on overall mAb2 production, whatever the scale, while having the advantage of at least reducing precipitation of the concentrated main feed.

Conclusion: Example 3 confirm the findings of examples 1 and 2 and highlight that a main feed at lower pH compared to its standard pH (i.e. pH 6.5), can be added to cell cultures during large scale process to produce an antibody without having adverse effects on the overall process performances and could reduce and/or avoid the precipitation of the main feed during the overall cultivation process.

### Example 4—Supplementation of Non-Concentrated Main Feed at Low pH to Produce mAb3

For this experiment, 2 L bioreactors have been inoculated with CHO cells producing mAb3 at a seeding density of 0.35×10⁶ cells/mL. In this experiment, two experimental conditions were tested in fed-batch process at 2 L scales as described in the above experimental procedures. Bioreactor ID 10/11 were fed with non-concentrated main feeds according to the same feeding strategy but at two different pH: respectively 6.5 and 5.5 (see Table 5).

TABLE 5
Experimental conditions for example 4

| Conditions | pH Main feed | Scale |
|---|---|---|
| Bioreactor ID 10 | 6.5 | 2 L |
| Bioreactor ID 11 | 5.5 | 2 L |

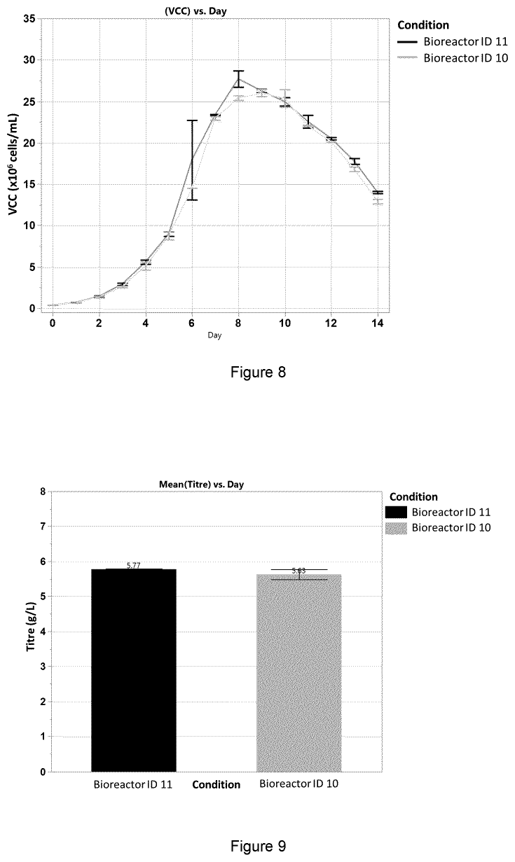

Cell growth profiles are shown in FIG. 8. Cell growth for both conditions presented similar trend until day 14. Bioreactor ID 10 and 11 shows a comparable titre (on day 14) (See FIG. 9). The results confirm that there is no negative impact of adding a non-concentrated main feed at lower pH on overall mAb3 production. Visual examinations were made at the end of the N stage (after disconnection of the main feed bottle from the bioreactors 10/11; pictures not shown). Cloudiness of the precipitated non-concentrated main feed solution was observed at pH 6.5. On the contrary, a clear/limpid solution was observed for the non-concentrated main feed solution at pH 5.5.

TABLE 6
Precipitation occurrence inside main feed bottle used
for supplementation during N stage

| | | Precipitation in the main |
|---|---|---|
| Conditions | pH main feed | feed bottle |
| Bioreactor ID 10 | 6.5 | Yes |
| Bioreactor ID 11 | 5.5 | No |

(Yes = precipitate occurrence; No = no precipitate occurrence).

Conclusion: Example 4 confirm the findings of examples 1, 2, 3 and highlight that a main feed at lower pH compared to its standard pH (i.e. pH 6.5), can be added to cell cultures to produce an antibody without having adverse effects on the overall process performances. The example 4 highlight that lowering pH for non-concentrated feed could reduce and/or avoid the precipitation of a non-concentrated main feed during the overall cultivation process.

### Example 5—Supplementation of Main Feed at Low pH to Produce mAb4

For this experiment, 2 L bioreactors have been inoculated with CHO cells producing mAb4 at a seeding density of 7.5×10⁶ cells/mL. In this experiment, two experimental conditions were tested in fed-batch process at 2 L scales as described in the above experimental procedures. Bioreactor ID 12/13 had the same feeding strategy but were fed with concentrated main feeds having two different pH: respectively 6.0 and 5.5 (see Table 7).

TABLE 7
Experimental conditions for example 5

| Conditions | pH Main feed | Scale |
|---|---|---|
| Bioreactor ID 12 | 6.0 | 2 L |
| Bioreactor ID 13 | 5.5 | 2 L |

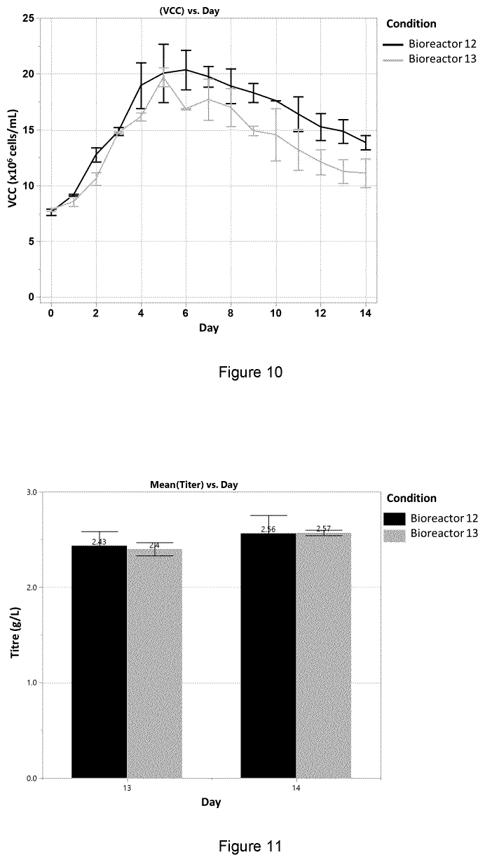

Although Bioreactor 13 displayed a slightly lower cell growth compared to Bioreactor ID 12 (see FIG. 10), they presented a comparable titre (on day 14) (See FIG. 11). The results confirm that there is no negative impact of adding a main feed at lower pH on overall mAb4 production. Visual examinations were made at the end of the N stage (after disconnection of the main feed bottle from the bioreactors 12/13; pictures not shown). A clear/limpid solution was observed for the main feed solution at pH 6.0 and pH 5.5.

TABLE 8
Precipitation occurrence inside main feed bottle used
for supplementation during N stage

| | | Precipitation in the main |
|---|---|---|
| Conditions | pH main feed | feed bottle |
| Bioreactor ID 12 | 6.0 | No |
| Bioreactor ID 13 | 5.5 | No |

(Yes = precipitate occurrence; No = no precipitate occurrence).

Conclusion: Example 5 confirm the findings of examples 1, 2, 3 and 4 and highlight that a main feed at lower pH, can be added to cell cultures to produce an antibody without having detrimental effect on the overall process performances. Lowering pH by 0.5 pH unit (i. e. pH main feed 5.5 compared to pH main feed 6.0) seems to have a minimal impact on cell growth. The example 5 highlight the feasibility of supplementing low pH for main feed over a 14-days of a cell culture.

### REFERENCES

Kshirsagar R., et al. (2012) Biotech. and Bioeng., 109:10, 2523-2532
US20130281355
WO2013158275
WO2011134919
Hecklau C, et al. (2016) J Biotech, 218:53-63
Zang L. et al. (2011) Anal. Chem, 83:5422-5430
WO2008013809
WO2008141207
WO2011133902
WO9808934
US20060148074
WO2015197772

1-14. (canceled)

15. A process for producing a recombinant protein, wherein the process comprises the steps of culturing mammalian cells expressing said recombinant protein in a culture medium, supplementing the cell culture during a production phase with at least one feed medium, wherein the pH of this at least one feed medium is from about 5.0 to about 6.3.

16. The process according to claim 15, wherein the at least one feed medium is a main feed medium or a concentrated main feed medium.

17. The process according to claim 15, wherein the pH is from about 5.2 to about 6.2.

18. The process according to claim 15, wherein the process is a fed batch process.

19. The process according to claim 15, wherein the feed medium does not comprise any one of the free amino acids Cys and Tyr.

20. The process according to claim 15, wherein the feed medium does not comprise any one of the free amino acids Cys, Tyr and Trp.

21. The process according to claim 15, wherein the mammalian cells are Chinese Hamster Ovary (CHO) cells.

22. The process according to claim 15, wherein the recombinant protein is a cytokine, a growth factor, a hormone, an antibody or a fusion protein.

23. The process according to claim 22, wherein the antibody is a chimeric antibody, a humanised antibody or a fully human antibody

24. The process according to claim 22, wherein the antibody is an IgG1, IgG2, IgG3 or IgG4.

25. A feed medium for use in the process according to claim 15, wherein the pH of this at least one feed medium is from about 5.0 to about 6.3.

26. A recombinant protein produced by the process according to claim 15.

27. A process for reducing or preventing precipitation in a feed medium, wherein the process comprises the steps of culturing mammalian cells expressing a recombinant protein in a culture medium, and supplementing the cell culture during a production phase with at least one feed medium, wherein the pH of this at least one feed medium is from about 5.0 to about 6.3.

28. A process for culturing mammalian cells expressing a recombinant protein, wherein the process comprises the steps of culturing the mammalian cells in a culture medium and supplementing the cell culture during a production phase with at least one feed medium, wherein the pH of this at least one feed medium is from about 5.0 to about 6.3.
